(19) United States
(12) Patent Application Publication
(10) Pub. No.: US 2007/0196911 A1
(43) Pub. Date: Aug. 23, 2007

(54) Devices and methods for growing human cells

(76) Inventors: Giovanni Mambrini, Mirandola (IT); Giuseppe Astori, Treviso (IT); Ivo Panzani, Mirandola (IT); Leonardo Bigi, Mirandola (IT); Valentina Adami, Udine (IT); Walter Malangone, Udine (IT); Elisabetta Falasca, Udine (IT)

Correspondence Address: POPOVICH, WILES & O'CONNELL, PA;650 THIRD AVENUE SOUTH, SUITE 600, MINNEAPOLIS, MN, 55402, US

(21) Appl. No.: 11/651,919

(22) Filed: Jan. 10, 2007

Related U.S. Application Data
(63) Continuation of application No. PCT/EP04/07689, filed on Jul. 12, 2004.

(51) Int. Cl. C12M 1/24; C12N 5/08

(52) U.S. Cl. 435/297.5; 435/304.3; 435/372

(57) ABSTRACT

A bioreactor having: a reaction chamber; a first port adapted for the introduction of human cells; a second port adapted for gas exchange, the second port comprising a filter; a third port adapted for introducing culture medium; a fourth port adapted for sampling cells; and a fifth port adapted for harvesting the human cells after they have been cultured.

This application is a continuation of International Application No. PCT/EP04/007689, filed Jul. 12, 2004, the contents of which are hereby incorporated herein by reference.

### FIELD OF THE INVENTION

The present invention relates to devices and methods for growing human cells, especially hematopoietic cells.

### BACKGROUND OF THE INVENTION

Hematopoietic cells are produced in the bone marrow from a totipotent stem cell which is able to reproduce itself and give rise to all the other hematopoietic cells. This stem cell gives rise to progenitor cells, for example, erythroid progenitors and myeloid progenitors, which are committed to differentiate into specific types of cells. Progenitor cells give rise to differentiated cells which have a limited or no capacity to proliferate. In humans, stem cells and progenitor cells express the CD34 antigen, while more differentiated hematopoietic cells do not.

Hematopoietic cells are derived from bone marrow, peripheral blood, or umbilical cord blood of a patient or a suitable donor. These cells can be used to reconstitute the patient's blood-clotting and infection-fighting functions when these have been compromised by, for example, chemotherapy.

Umbilical cord blood from unrelated donors is increasingly used as a source of hematopoietic cells for allogenic transplantation after myeloablative therapy. In spite of several advantages of using umbilical cord blood, the use is restricted by the limited number of cells as compared to cells available from bone marrow or peripheral blood.

It is desirable to provide a way to increase the number of umbilical cord blood cells which could then be used when treating adult patients. U.S. Pat. No. 5,635,387 describes methods for growing hematopoietic cells. The '387 patent describes methods for growing hematopoietic cells without stromal cells layers or stromal cell conditioned medium, which was believed essential in the early development of this art. Current expansion procedures are performed in cell expansion chambers or cell expansion bags that are not fully dedicated to this purpose. Not using dedicated systems causes several problems. For instance, most systems are not closed systems meaning that human stem cells expansion requires skilled personnel and expensive equipment to perform a successful expansion procedure. The open circuits, even when used by skilled personnel and well equipped labs, do not insure the sterility of the final product and do not meet the sterile handling procedures required provided by the current regulatory recommendations and/or guidelines. The available closed circuit systems provide non-dedicated systems that are not specifically designed for human stem cells expansion; furthermore, such systems require complex, expensive apparatuses to operate.

Most stem cell expansion procedures are currently performed using reagents that contain animal-derived products. The use of animal-derived products does not allow clinical use of the expanded cell populations. Expansion of hematopoietic stem cells is currently performed using different media not specifically designed for hematopoietic stem cell expansion. In particular, reagents are not made of a defined media and a mixture of growth factors dedicated to hematopoietic stem cells expansion. Reagents currently in use have different concentrations, combinations of growth factors, often do not meet specific regulatory requirements such as apirogenicity, are not user-friendly, and are difficult to use without expensive equipment and skilled personnel.

There remains a need in the art for improved devices and methods of culturing human hematopoietic stem cells which result in the expansion of the number of these cells and produces good recovery of the cells while maintaining sterility and without compromising the viability of the cells.

The invention described herein provides a device that can be used for culturing human hematopoietic stem cells. However, the device can also be used to culture other human cells, including other types of human stem cells, human muscle cells, human skin cells, etc.

### SUMMARY OF THE INVENTION

The invention provides a bioreactor comprising: a reaction chamber; a first port adapted for the introduction of cells; a second port adapted for gas exchange, the second port comprising a filter; a third port adapted for introducing culture medium; a fourth port adapted for sampling cells; and a fifth port adapted for harvesting the cells after they have been cultured.

The invention provides a method for increasing the number of human hematopoietic cells in vitro comprising: providing a bioreactor described above; introducing human hematopoietic cells into the first port; introducing culture medium through the third port; and culturing the cells under conditions and for a time sufficient to increase the number of cells.

The invention provides a method for increasing the number of human hematopoietic CD34-positive cells in vitro, comprising: providing CD34-positive human hematopoietic cells; inoculating the CD34-positive cells at an initial density of from 1×10⁴ to 5×10⁶ cells/ml into a bioreactor containing a culture medium comprising a nutrient medium and growth factors effective for expansion of CD34-positive cells, wherein the growth factors comprise Flt-3L, thrombopoietin, interleukin-3, and stem cell factor; and culturing the CD34-positive cells under conditions and for a time sufficient to increase the number of CD34-positive cells.

Additional features and advantages of the invention are set forth in the description which follows and in part will be apparent from the description. The objectives and other advantages of the invention will be realized and attained by the devices and methods for growing human cells as particularly pointed out in the written description and claims.

It is to be understood that both the foregoing general description and the following detailed description are exemplary and explanatory and are intended to provide further explanation of the invention as claimed.

### BRIEF DESCRIPTION OF THE DRAWINGS

FIG. 1 shows a perspective view of a bioreactor of the invention.

FIG. 2 shows a top view of the bioreactor of FIG. 1.

FIG. 3 shows a top view of tubing and sterile bags that can be used with a bioreactor of the invention.

### DETAILED DESCRIPTION OF THE INVENTION

The invention provides a bioreactor comprising: a reaction chamber; a first port adapted for the introduction of cells; a second port adapted for gas exchange, the second port comprising a filter; a third port adapted for introducing culture medium; a fourth port adapted for sampling cells; and a fifth port adapted for harvesting the cells after they have been cultured. In an embodiment of the invention, the filter of the second port is a gas permeable membrane filter. In another embodiment of the invention, the third port comprises a filter. In yet another embodiment of the invention, the bioreactor further comprises a sixth port adapted for introducing culture medium, the sixth port comprising a filter.

In an embodiment of the invention, the first port comprises a pierceable cap. In another embodiment of the invention, the fourth port comprises a pierceable cap. In an embodiment of the invention, the bioreactor is adapted to harvest the cells after they have been cultured by draining the cells out of the fifth port.

In an embodiment of the invention, the reaction chamber has an expansion surface of from 25 cm² to 600 cm². In another embodiment of the invention, the reaction chamber has an expansion surface of from 150 cm² to 250 cm². In an embodiment of the invention, the reaction chamber is made of clear, tissue culture-treated plastic.

In an embodiment of the invention, the bioreactor comprises a label near the first port indicating that cells can be introduced through the first port. In another embodiment of the invention, the bioreactor comprises a label near the second port indicating that gas can exchanged through the second port. In yet another embodiment of the invention, the bioreactor comprises a label near the third port indicating that culture medium can be introduced through the third port. In an embodiment of the invention, the bioreactor comprises a label near the fourth port indicating that the contents of the reaction chamber can be sampled through the fourth port. In another embodiment of the invention, the bioreactor comprises: (i) a label near the third port indicating that culture medium can be introduced through the third port at day zero, and (ii) a label near the sixth port indicating that culture medium can be introduced through the sixth port at day seven. All of the labels can be attached to the bioreactor or molded into the bioreactor.

The bioreactor can be used for culturing human cells, including human hematopoietic stem cells, other types of human stem cells, human muscle cells, human skin cells, etc.

The invention provides a kit comprising a bioreactor described herein and tubing connected to the fifth port. In an embodiment of the invention, the kit further comprises additional tubing and one or more sterile bags. In another embodiment of the invention, the kit further comprises a nutrient medium in a first container, and the growth factors Flt-3L, thrombopoietin, interleukin-3, and stem cell factor in a second container. In another embodiment of the invention, the growth factors are present in the following concentrations: Flt-3L at 1.9 micrograms/milliliter; thrombopoietin at 1.9 micrograms/milliliter; interleukin-3 at 0.17 micrograms/milliliter; and stem cell factor at 1 micrograms/milliliter.

The invention provides a method for increasing the number of human hematopoietic cells in vitro comprising: providing a bioreactor described herein; introducing human hematopoietic cells into the first port; introducing culture medium through the third port; and culturing the cells under conditions and for a time sufficient to increase the number of cells. In an embodiment of the invention, the cells are harvested through the fifth port after they have been cultured. In another embodiment of the invention, the bioreactor further comprises a sixth port adapted for introducing culture medium, the sixth port comprising a filter, and seven days after culture medium has been introduced through the third port, culture medium is introduced through the sixth port.

In an embodiment of the invention, the human hematopoietic cells are CD34-positive. In another embodiment of the invention, the culture medium comprises a nutrient medium and growth factors effective for expansion of human hematopoietic cells, wherein the growth factors comprise Flt-3L, thrombopoietin, interleukin-3, and stem cell factor. In embodiments of the invention, the human hematopoietic cells are derived from human umbilical cord blood, human bone marrow, or human peripheral blood.

The invention provides a method for increasing the number of human hematopoietic cells in vitro, comprising: providing CD34-positive human hematopoietic cells; inoculating the CD34-positive cells at an initial density of from 1×10⁴ to 5×10⁶ cells/ml into a bioreactor containing a culture medium comprising a nutrient medium and growth factors effective for expansion of CD34-positive cells, wherein the growth factors comprise Flt-3L, thrombopoietin, interleukin-3, and stem cell factor; and culturing the CD34-positive cells under conditions and for a time sufficient to increase the number of CD34-positive cells. In an embodiment of the invention, the CD34-positive cells are derived from human umbilical cord blood, human bone marrow, or human peripheral blood. In another embodiment, the CD34-positive cells are inoculated into the bioreactor at an initial cells number from 5×10⁵ to 1.5×10⁶ cells. In yet another embodiment, the bioreactor has an expansion surface of from 25 cm² to 600 cm².

In an embodiment of the invention, the number of CD34-positive human hematopoietic cells increases at least three-fold. In another embodiment, the hematopoietic growth factors consist essentially of Flt-3L, thrombopoietin, interleukin-3, and stem cell factor. In another embodiment, subsequent to the step of culturing, the human hematopoietic cells are harvested from the culture medium. In embodiments of the invention, the CD34-positive cells are cultured for from four to twenty days. In yet another embodiment, the CD34-positive cells are cultured for seven days, and then on the seventh day, additional nutrient medium and growth factors are added, the CD34-positive cells are cultured for five more days, and then harvested.

In an embodiment of the invention, the growth factors consist essentially of Flt-3L, thrombopoietin, interleukin-3, and stem cell factor, and these growth factors are present in the following concentrations at the beginning of the culturing step: Flt-3L at 0.01 to 0.1 micrograms/milliliter; thrombopoietin at 0.01 to 0.1 micrograms/milliliter; interleukin-3 at 0.001 to 0.01 micrograms/milliliter; and stem cell factor at 0.01 to 0.1 micrograms/milliliter. In another embodiment, the growth factors consist essentially of Flt-3L, thrombopoietin, interleukin-3, and stem cell factor, and these growth factors are present in the following concentrations at the beginning of the culturing step: Flt-3L at 0.05 micrograms/milliliter; thrombopoietin at 0.05 micrograms/milliliter; interleukin-3 at 0.0043 micrograms/milliliter; and stem cell factor at 0.025 micrograms/milliliter. In another embodiment, the culture medium and bioreactor do not contain stromal cells or stromal cell conditioned medium.

The invention provides a reagent consisting essentially of the growth factors Flt-3L, thrombopoietin, interleukin-3, and stem cell factor, and these growth factors are present in the following concentrations: Flt-3L at 1.9 micrograms/milliliter; thrombopoietin at 1.9 micrograms/milliliter; interleukin-3 at 0.17 micrograms/milliliter; and stem cell factor at 1 micrograms/milliliter.

The invention provides a kit comprising a bioreactor, a nutrient medium in a first container, and the growth factors Flt-3L, thrombopoietin, interleukin-3, and stem cell factor in a second container. In one embodiment, the kit further comprises one or more syringes. In another embodiment, the kit further comprises tubing and one or more sterile bags. In yet another embodiment of the kit, the growth factors are present in the following concentrations: Flt-3L at 1.9 micrograms/milliliter; thrombopoietin at 1.9 micrograms/milliliter; interleukin-3 at 0.17 micrograms/milliliter; and stem cell factor at 1 micrograms/milliliter.

In an embodiment of this invention, human cells are grown from umbilical cord blood in a bioreactor. After a suitable incubation period in nutrient and growth media, the cells are collected, washed to remove residual reagents and then made available for use.

The culture medium used to produce hematopoietic cells contains nutrient media and growth factors (cytokines). Various nutrient media and growth factors may be employed for the growth of hematopoietic cells. A suitable nutrient medium for this invention includes X-VIVO 20 (commercially available from Cambrex, East Rutherford, N.J.), or other serum-free media. The nutrient medium may be supplemented with 1 to 20% autologous plasma or heterologous plasma.

Growth factors or cytokines that may be included in the nutrient medium include human Flt-3L, thrombopoietin (TPO), interleukin 3 (IL-3), stem cell factor (SCF), human GM-CSF (granulocyte macrophage-colony stimulating factor) and G-CSF (granulocyte-colony stimulating factor), interleukins 1, 2, and 4 to 7, and erythropoietin.

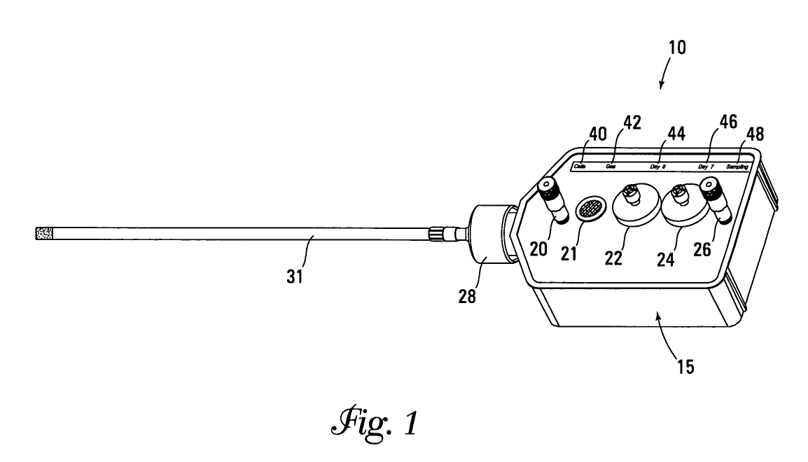

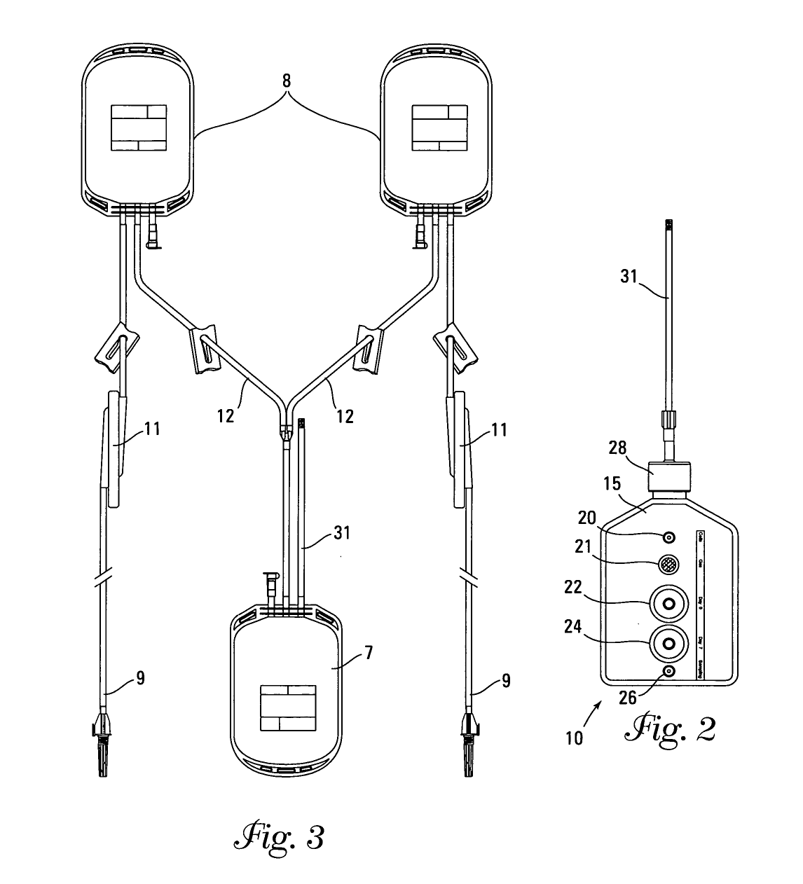

FIGS. 1 and 2 illustrate an embodiment of this invention, in which bioreactor 10 comprises reaction chamber 15. The chamber has a convenient shape that allows for distribution of culture medium and promotes cell growth. The chamber comprises a transparent polymeric material that is compatible with or has been treated to be compatible with biological materials.

Bioreactor 15 is provided with five ports (20, 21, 22, 24, 26). Port 20 (having label 40 “Cells”) has a pierceable cap to inject the cells. Port 21 (having label 42 “Gas”) has a filter for gas exchange with the outside environment. Port 22 (having label 44 “Day 0”) has a filter for injection of culture medium and growth factors at the beginning of procedure. Port 24 (having label 46 “Day 7”) has a filter for injection of culture medium and growth factors in subsequent days, such as day 7. Port 26 (having label 48 “Sampling”) has a pierceable cap to sample the cells.

Through ports 22 and 24 culture medium is delivered via syringe and through port 21 gases are exchanged. The gases exchanged include oxygen and carbon dioxide (CO₂). Preferably, the CO₂ content is controlled to a desired level, e.g., 5 percent. Physiologic temperatures are used to incubate the contents of the bioreactor, i.e., preferably 37° C., although the temperature may range from 25° C. to 37° C. Humidity is preferably kept at about 100 percent. Once incubation is complete, the hematopoietic cells exit the bioreactor via exit port 28, through tubing line 31, which can be connected to collection bag 7, via tubing 31, by means of a common sterile docking system. Bioreactor 15 is tilted toward port 28, and the cells flow into the collection bag 7 (shown in FIG. 3). In a preferred embodiment, the culture period ranges from 10 to 14 days. Bags 8, which are preconnected to the collection bag 7, can be loaded with washing saline solution through lines 9. Washing solution can be introduced into the collection bay 7 through tubing 12. Sterility of the washing liquids is assured by sterile filters 11.

In one embodiment of this invention, hematopoietic cells are produced by first introducing X-VIVO 20 nutrient medium and growth factors into the reactor via port 22. The growth factors include IL3, TPO, SCF, and Flt3-L. Selected CD34+ cells are injected into the chamber via port 20, and the bioreactor is placed into an incubator at physiologic temperatures under controlled atmosphere. After a desired incubation period, additional X-VIVO 20 nutrient medium and growth factors (as above) are added through port 24.

The bioreactor is then returned to the incubator. At the end of the second incubation time, the bioreactor is removed from the incubator, and the cells are collected in the collection bag.

The reagents used in this invention typically are stored at 4° C. Preferably, the reagents are provided as two cytokine mix vials (CK-mix A and CK-mix B) and two culture medium vials (MED A and MED B). The contents of the “A” vials are used on Day 0 to inoculate the chamber and the contents of the “B” vials are added to the chamber on Day 7. Syringes are used to introduce the reagents into the reactor.

### EXAMPLE

A bioreactor is prepared by using a commercially available polystyrene tissue culture flask (e.g., code 35-3028 from Becton Dickinson Labware, Franklin Lakes, N.J., USA), equipped as follows: (1) five ports are provided on the upper portion of the flask. Two ports have polyethersulfone (PES) 0.2 micrometer filters. Two ports have a perforable connector; one port has a gas permeable filtering membrane; (2) a sixth port connects the tissue culture flask to a 500 ml sterile bag for collection of cells at the end of the culture period. The bioreactor has an expansion surface of about 175 cm² of tissue culture treated polystyrene.

At day 0, a mixture of nutrient medium and growth factors is introduced with a sterile syringe through a port having a 0.2 micrometer (μm) sterilizing filter. The mixture of nutrient medium and growth factors is prepared by mixing a cytokine composition (CK-mix A) containing 0.270 μg (micrograms) IL3, 3.0 μg TPO, 1.5 μg SCF, and 3.0 μg Flt3-L suspended in 1560 microliters of nutrient medium and 60 ml of X-VIVO 20 medium (MED A). At day 0 and following the inoculation of nutrient medium and growth factors, CD34+ selected cells are inoculated in the bioreactor using another dedicated port. E.g., 1.2×10⁶ selected cells are inoculated with 61.5 ml of nutrient medium (initial cells concentration of approx. 20,000 cells/ml). The CD34+ cells have a minimum viability of 80 percent and a minimum purity of 70 percent.

The contents of the bioreactor are incubated at 37° C., at about 100% humidity, in an air atmosphere containing about 5% CO₂. After one week of culture or incubation (i.e., Day 7), a mixture of nutrient medium and growth factors is introduced with a sterile syringe through a another dedicated port having a 0.2 micrometer (μm) sterilizing filter. The mixture of nutrient medium and growth factors is prepared by mixing a cytokine composition (CK-mix B) containing 0.135 μg (micrograms) IL3, 1.5 μg TPO, 0.75 μg SCF, and 1.5 μg Flt3-L suspended in 776 microliters of the nutrient medium and 30 ml of X-VIVO 20 medium (MED B).

At the end of the culture period, the contents of the bioreactor are drained out of the bioreactor through a dedicated port and flow into a sterile bag. Residual cytokines are removed by washing the cells by standard means.

This method typically produces a CD34+ expansion of three to forty-fold and a total number of cells expansion of 30 to 300-fold, averaging about 200-fold. The vitality of the cells is greater than 80 percent.

The above description and accompanying drawings are provided for the purpose of describing embodiments of the invention and are not intended to limit the scope of the invention in any way. It will be apparent to those skilled in the art that various modifications and variations can be made in the devices and methods for growing cells without departing from the spirit or scope of the invention. Thus, it is intended that the present invention cover the modifications and variations of this invention provided they come within the scope of the appended claims and their equivalents.

What is claimed is:

1. A bioreactor comprising:
a reaction chamber;
a first port adapted for the introduction of human cells;
a second port adapted for gas exchange, the second port comprising a filter;
a third port adapted for introducing culture medium;
a fourth port adapted for sampling cells; and
a fifth port adapted for harvesting the human cells after they have been cultured.

2. A bioreactor of claim 1, wherein the filter of the second port is a gas permeable membrane filter.

3. A bioreactor of claim 1, wherein the third port comprises a filter.

4. A bioreactor of claim 1, wherein the bioreactor further comprises a sixth port adapted for introducing culture medium, the sixth port comprising a filter.

5. A bioreactor of claim 1, wherein the first port comprises a pierceable cap.

6. A bioreactor of claim 1, wherein the fourth port comprises a pierceable cap.

7. A bioreactor of claim 1, wherein the bioreactor is adapted to harvest the human cells after they have been cultured by draining the cells out of the fifth port.

8. A bioreactor of claim 1, wherein the reaction chamber has an expansion surface of from 25 cm² to 600 cm².

9. A bioreactor of claim 1, wherein the reaction chamber has an expansion surface of from 150 cm² to 250 cm².

10. A bioreactor of claim 1, wherein the reaction chamber is made of clear, tissue culture-treated plastic.

11. A bioreactor of claim 1, wherein the bioreactor comprises a label near the first port indicating that cells can be introduced through the first port.

12. A bioreactor of claim 1, wherein the bioreactor comprises a label near the second port indicating that gas can exchanged through the second port.

13. A bioreactor of claim 1, wherein the bioreactor comprises a label near the third port indicating that culture medium can be introduced through the third port.

14. A bioreactor of claim 1, wherein the bioreactor comprises a label near the fourth port indicating that the contents of the reaction chamber can be sampled through the fourth port.

15. A bioreactor of claim 4, wherein the bioreactor comprises: (i) a label near the third port indicating that culture medium can be introduced through the third port at day zero, and (ii) a label near the sixth port indicating that culture medium can be introduced through the sixth port at day seven.

16. A bioreactor of claim 11, wherein the label is attached to the bioreactor or is molded into the bioreactor.

17. A kit comprising a bioreactor of claim 1 and tubing connected to the fifth port.

18. A kit of claim 17, further comprising additional tubing and one or more sterile bags.

19. A kit of claim 17, further comprising a nutrient medium in a first container, and the growth factors Flt-3L, thrombopoietin, interleukin-3, and stem cell factor in a second container.

20. A kit of claim 19, wherein the growth factors are present in the following concentrations:

| | |
|---|---|
| Flt-3L | 1.9 micrograms/milliliter; |
| thrombopoietin | 1.9 micrograms/milliliter; |
| interleukin-3 | 0.17 micrograms/milliliter; and |
| stem cell factor | 1 micrograms/milliliter. |

21. A method for increasing the number of human hematopoietic cells in vitro comprising:
providing a bioreactor of claim 1;
introducing human hematopoietic cells into the first port;
introducing culture medium through the third port; and
culturing the cells under conditions and for a time sufficient to increase the number of cells.

22. A method of claim 21, wherein the cells are harvested through the fifth port after they have been cultured.

23. A method of claim 21, wherein the bioreactor further comprises a sixth port adapted for introducing culture medium, the sixth port comprising a filter, and seven days after culture medium has been introduced through the third port, culture medium is introduced through the sixth port.

24. A method of claim 21, wherein the human hematopoietic cells are CD34-positive.

25. A method of claim 21, wherein the culture medium comprises a nutrient medium and growth factors effective for expansion of human hematopoietic cells, wherein the growth factors comprise Flt-3L, thrombopoietin, interleukin-3, and stem cell factor.

26. A method of claim 21, wherein the human hematopoietic cells are derived from human umbilical cord blood.

27. A method of claim 21, wherein the human hematopoietic cells are derived from human bone marrow.

28. A method of claim 21, wherein the human hematopoietic cells are derived from human peripheral blood.

29. A method for increasing the number of human hematopoietic cells in vitro, comprising:
providing CD34-positive human hematopoietic cells;
inoculating the CD34-positive cells at an initial density of from 1×10⁴ to 5×10⁶ cells/ml into a bioreactor containing a culture medium comprising a nutrient medium and growth factors effective for expansion of CD34-positive cells, wherein the growth factors comprise Flt-3L, thrombopoietin, interleukin-3, and stem cell factor; and
culturing the CD34-positive cells under conditions and for a time sufficient to increase the number of CD34-positive cells.

30. A method of claim 29, wherein the CD34-positive cells are derived from human umbilical cord blood.

31. A method of claim 29, wherein the CD34-positive cells are derived from human bone marrow.

32. A method of claim 29, wherein the CD34-positive cells are derived from human peripheral blood.

33. A method of claim 29, wherein the CD34-positive cells are inoculated into the bioreactor at an initial density of from 1×10⁴ to 5×10⁶ cells/ml.

34. A method of claim 29, wherein the bioreactor has an expansion surface of from 25 cm² to 600 cm².

35. A method of claim 29, wherein the number of CD34-positive human hematopoietic cells increases at least three-fold.

36. A method of claim 29, wherein the number of CD34-positive human hematopoietic cells increases at least five-fold.

37. A method of claim 29, wherein the growth factors consist essentially of Flt-3L, thrombopoietin, interleukin-3, and stem cell factor.

38. A method of claim 29, further comprising, subsequent to the step of culturing, harvesting the human hematopoietic cells from the culture medium.

39. A method of claim 29, wherein the CD34-positive cells are cultured for from four to twenty days.

40. A method of claim 29, wherein the CD34-positive cells are cultured for seven days, and then on the seventh day, additional nutrient medium and growth factors are added, the CD34-positive cells are cultured for five more days, and then harvested.

41. A method of claim 29, wherein the growth factors consist essentially of Flt-3L, thrombopoietin, interleukin-3, and stem cell factor, and these growth factors are present in the following concentrations at the beginning of the culturing step:

| | |
|---|---|
| Flt-3L | 0.01 to 0.1 micrograms/milliliter; |
| thrombopoietin | 0.01 to 0.1 micrograms/milliliter; |
| interleukin-3 | 0.001 to 0.01 micrograms/milliliter; and |
| stem cell factor | 0.01 to 0.1 micrograms/milliliter. |

42. A method of claim 29, wherein the growth factors consist essentially of Flt-3L, thrombopoietin, interleukin-3, and stem cell factor, and these growth factors are present in the following concentrations at the beginning of the culturing step:

| | |
|---|---|
| Flt-3L | 0.05 micrograms/milliliter; |
| thrombopoietin | 0.05 micrograms/milliliter; |
| interleukin-3 | 0.0043 micrograms/milliliter; and |
| stem cell factor | 0.025 micrograms/milliliter. |

43. A method of claim 29, wherein the culture medium and bioreactor do not contain stromal cells or stromal cell conditioned medium.

44. A reagent consisting essentially of the growth factors Flt-3L, thrombopoietin, interleukin-3, and stem cell factor, and these growth factors are present in the following concentrations:

| | |
|---|---|
| Flt-3L | 1.9 micrograms/milliliter; |
| thrombopoietin | 1.9 micrograms/milliliter; |
| interleukin-3 | 0.17 micrograms/milliliter; and |
| stem cell factor | 1 micrograms/milliliter. |

45. A kit comprising a bioreactor, a nutrient medium in a first container, and the growth factors Flt-3L, thrombopoietin, interleukin-3, and stem cell factor in a second container.

46. A kit of claim 45, further comprising tubing and one or more sterile bags.

47. A kit of claim 45, wherein the growth factors are present in the following concentrations:

| | |
|---|---|
| Flt-3L | 1.9 micrograms/milliliter; |
| thrombopoietin | 1.9 micrograms/milliliter; |
| interleukin-3 | 0.17 micrograms/milliliter; and |
| stem cell factor | 1 micrograms/milliliter. |

48. A method for increasing the number of human cells in vitro comprising:
providing a bioreactor of claim 1;
introducing human cells into the first port;
introducing culture medium through the third port; and
culturing the cells under conditions and for a time sufficient to increase the number of cells.

49. A method of claim 48, wherein the cells are harvested through the fifth port after they have been cultured.

50. A method of claim 48, wherein the bioreactor further comprises a sixth port adapted for introducing culture medium, the sixth port comprising a filter, and seven days after culture medium has been introduced through the third port, culture medium is introduced through the sixth port.
